(19) United States
(12) Patent Application Publication
(10) Pub. No.: US 2005/0003467 A1
(43) Pub. Date: Jan. 6, 2005

(54) Tumor necrosis agent

(75) Inventor: Katsuyoshi Hori, Sendai-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 10/846,514

(22) Filed: May 17, 2004

Related U.S. Application Data
(63) Continuation of application No. PCT/JP02/11918, filed on Nov. 15, 2002.

(30) Foreign Application Priority Data
Nov. 16, 2001 (JP) 2001-352188

(51) Int. Cl. G01N 33/574

(52) U.S. Cl. 435/7.23

(57) ABSTRACT

The present invention relates to novel drugs (pharmaceuticals) such as tumor necrosis agents, uses of substances having tumor necrosis effect for production of tumor necrosis agents, methods of screening substances having tumor necrosis effect, and methods of treating or improving tumor(s), particularly methods of necrotizing tumor(s). The invention further provides active ingredients that are used in drugs suitable for destroying or damaging tumor(s) effectively and selectively to animal needing treatment of tumor(s), particularly humans (patients), and enables the use of the active ingredients in drugs, the screening of such active ingredients and the therapy of tumor(s) using such drugs.

### CROSS-REFERENCE TO RELATED APPLICATIONS

The present application is a continuation of International Application PCT/JP02/11918, filed on Nov. 15, 2002, which claims priority to Japanese Application No. JP 2001-352188, filed on Nov. 16, 2001, which are hereby incorporated by reference in therein entirety.

### BACKGROUND OF THE INVENTION

1. Field of the Invention

The present invention provides tumor necrosis agents that selectively destroy or damage tumor tissues with little or no side effects to normal tissues by using, as active ingredients, substances having the following effects in the administration to a tumor-bearing living body,

a. an effect of contracting blood vessels that feed nutrients to tumor tissues, especially blood vessels dominating tumor blood flow strongly and persistently, and

b. an effect of hemolyzing red blood cells (erythrocytes) in blood vessels on peripheral portion of the tumor tissues.

Also provided by the present invention are methods of screening substances (active ingredients) having these effects, uses of the active ingredients for production of drug(s), methods of using the drug(s), methods of treating or improving tumor(s) in particular, and the like.

2. Discussion of the Background

The objective of cancer chemotherapy lies in attacking and destroying all cancer cells that have grown in vivo by using anticancer agents. Accordingly, heretofore, anticancer agents have targeted cancer cells. However, chemical substances that show strong cytotoxicity to cancer cells also demonstrate the same effect on normal cells that actively divide, such as hemopoietic cells and digestive tract mucosal cells, resulting in severe side effects. Unfortunately, to date, the number of drugs showing selective cytotoxicity to tumor cells is very few.

Accordingly, the present invention seeks to meet the demand to develop drugs that selectively attack tumor tissues (cells) with little or no side effects. Specifically, the present invention seek to solve the problems existing in the art by providing a selective therapy of tumors with substantially diminished side effects by developing active ingredients for drugs (and the drugs themselves) which selectively attack the tumor tissues to reduce the size or to regress completely.

### SUMMARY OF THE INVENTION

It is an object of the present invention to provide a method of identifying a tumor necrosis agent having the following effects when administered to a tumor-bearing living body:

a. an effect of contracting a blood vessel that feeds a nutrient to a tumor tissue, and

b. an effect of hemolyzing red blood cells in a blood vessel on a peripheral portion of the tumor tissue,

where the method entails:

administering a sample containing a candidate compound to a tumor-bearing experimental animal; and

assessing the ability of the candidate compound to provide effects (a) and (b) as compared to a control group in which the candidate compound is not administered.

Within this object, it is preferred that the compound is not (Z)-N-[2-methoxy-5-[2-(3,4,5-trimethoxyphenyl)vinyl]phenyl]-L-serinamide hydrochloride.

In another object of the present invention, the aforementioned method further entails comparing effects (a) and (b) obtained for the candidate compound to those obtained for (Z)-N-[2-methoxy-5-[2-(3,4,5-trimethoxyphenyl)vinyl]phenyl]-L-serinamide hydrochloride; and

identifying candidate compounds in which at least one of effects (a) and (b) are equal to or greater than those obtained with (Z)-N-[2-methoxy-5-[2-(3,4,5-trimethoxyphenyl)vinyl]phenyl]-L-serinamide hydrochloride.

In yet another object of the present invention is to provide a tumor necrosis agent identified by the aforementioned method.

In still another object of the present invention is to provide a pharmaceutical composition containing the so-identified tumor necrosis agent and a pharmaceutically acceptable carrier.

Another object of the present invention is to provide a method of treating a tumor by administering to a subject in need thereof an effective amount of the aforementioned pharmaceutical composition.

In the aforementioned objects, a preferred compound is epinephrine.

An additional object of the present invention is to provide a method for contracting blood vessels (preferably, blood vessels that feed a nutrient to a tumor tissue) by administering to a patient in need thereof an effective amount of (Z)-N-[2-methoxy-5-[2-(3,4,5-trimethoxyphenyl)vinyl]phenyl]-L-serinamide hydrochloride.

Yet another object of the present invention is to provide a method for hemolyzing red blood cells (preferably, red blood cells in a blood vessel of a tumor tissue) by administering to a patient in need thereof an effective amount of (Z)-N-[2-methoxy-5-[2-(3,4,5-trimethoxyphenyl)vinyl]phenyl]-L-serinamide hydrochloride.

Another object of the present invention is to provide a method for clogging a blood vessel of a tumor tissue comprising administering to a patient in need thereof an effective amount of (Z)-N-[2-methoxy-5-[2-(3,4,5-trimethoxyphenyl)vinyl]phenyl]-L-serinamide hydrochloride

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows a micrograph of a tumor and an arteriole that feeds nutrients to the tumor tissue before administration of MTPVS. An arrow indicates an arteriole that feeds nutrients to the tumor tissue.

FIG. 2 shows a micrograph of a tumor and the arteriole that feeds nutrients to the tumor tissue 15 minutes after MTPVS administration.

FIG. 3 shows a micrograph of a tumor and an arteriole that feeds nutrients to the tumor tissue 2 hours after MTPVS administration.

FIG. 4 shows a micrograph of a tumor and an arteriole that feeds nutrients to the tumor tissue 2.5 hours after MTPVS administration. An inserted section on the left upper portion is an enlarged view of a blood vessel on peripheral portion of the tumor.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, the pharmaceutical sciences and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention provides a method of identifying a tumor necrosis agent having the following effects when administered to a tumor-bearing living body:

a. an effect of contracting a blood vessel that feeds a nutrient to a tumor tissue, and b. an effect of hemolyzing red blood cells in a blood vessel on a peripheral portion of the tumor tissue,

where the method entails:

administering a sample containing a candidate compound to a tumor-bearing experimental animal; and

assessing the ability of the candidate compound to provide effects (a) and (b) as compared to a control group in which the candidate compound is not administered.

The present inventors have discovered that identifying compounds by this method can solve the previously mentioned problems in the art regarding differential cytotoxicity. Specifically, substances having the following effects (functions) attack tumor tissues in experimental animals effectively and selectively to destroy or damage the tumors but have almost no side effects on normal tissues, and that the anti-tumor effect of the substances is not limited in the type of tumor or the site in which tumor occurs. Accordingly, the present invention is based on these findings:

a. an effect of contracting blood vessels that feed nutrients to tumor tissues, and

b. an effect of hemolyzing red blood cells (erythrocytes) in blood vessels in peripheral portion of the tumor tissues.

Examples of the substance having these effects include combretastatin derivatives or stilbene derivatives (refer to J. Med. Chem. 41:3022-3032, 1998; Bioorg. Med. Chem. Lett. 8: 3153-3158, 1998; Bioorg. Med. Chem. Lett. 8: 3371-3374, 1998; U.S. Pat. No. 56,122, U.S. Pat. No. 5,430,062, Japanese Patent Kokai Publications JP-A-7-228558 and JP-A-8-301831, PCT Publication WO 93/23357 and the like) having anti-tumor activity. It has also been confirmed that (Z)-N-[2-methoxy-5-[2-(3,4,5-trimethoxyphenyl)vinyl]phenyl]-L-serinamide hydrochloride (hereinafter referred to as “MTPVS”) has such effects.

In a particularly preferred embodiment of the present invention, the compound for use in the present invention is epinephrine.

That is, in one embodiment, the invention resides in drug(s) (hereinafter referred to also as “invention drug(s)”) such as tumor necrosis agent(s), comprising substance(s) having the following effects in the administration to tumor-bearing living body(ies):

a. an effect of contracting a blood vessel that feeds a nutrient to a tumor tissue, and

b. an effect of hemolyzing red blood cells (erythrocytes) in a blood vessel in a peripheral portion of the tumor tissue.

The effect of contracting a blood vessel is preferably an effect (function) of contracting a blood vessel supplying (dominating) to the tumor tissue strongly and persistently to decrease or stop the tumor blood flow. The effect of hemolyzing red blood cells is preferably an effect of hemolyzing red blood cells to clog the blood vessel of the tumor tissue.

The substance(s) exhibiting the foregoing effects can irreversibly block the blood flow of the tumor tissue to necrotize the tumor.

The detection of the foregoing effects can be performed readily using tumor (not limited in particular)-bearing experimental animals, for example, rodents such as rats. In this case, the route of administration to the experimental animals is not limited, and intravenous administration is advantageous because the desired effects can be detected at high sensitivity.

With respect to the evaluation of the foregoing effects, the effects of samples containing candidate compounds can easily be detected in comparison with those of a control group, for example, a physiological saline administration group. It is preferable that the effects of samples give a significant difference in comparison with those of the control group.

In a more preferable method, it is also possible to use substance(s) having one or more effects that are equal to or higher than those of MTPVS in the administration to rodents such as rats. As the method of evaluating such effects, a method is convenient in which samples are administered to experimental animals, for example, rodents such as rats and a tumor is observed through biomicroscope or histopathological section and compared with that of a control administration group.

In another embodiment, the invention resides in a use of a substance having the foregoing effects (a) and (b) in the administration to a tumor-bearing living body, for production of the tumor necrosis agent(s).

The invention is a method in which the active ingredient used in the invention drug(s) is used in the drug, and the active ingredient itself can be used as the drug(s). Further, preparations can be produced, as required, by using various carriers or additives for preparations (for example, for an anti-tumor agent) used so far. At this time, it can be stored in appropriate accommodation containers according to the properties or the dosage form of the active ingredient.

As stated above, the effect of contracting a blood vessel is preferably an effect of contracting supplying a blood vessel to a tumor tissue strongly and persistently to decrease or stop the tumor blood flow. The effect of hemolyzing red blood cells is preferably an effect of hemolyzing red blood cells to clog the blood vessel of the tumor tissue.

With respect to the tumor necrosis agent(s), any type of the tumor necrosis agents (invention drug(s)) described above can be used.

The report on the anti-tumor agents excellent in effectiveness and selectivity as in the invention is not found in anti-tumor agents known so far. However, such substances are included in known anti-tumor agents unless the degrees of the intended effects are queried. It is thus required to select and use the substances except the known anti-tumor agents or active ingredients (MTPVS and the like) thereof.

In still another embodiment, the invention resides in a method of screening a substance having a tumor necrosis effect, by administering samples in vivo to tumor-bearing experimental animals, then detecting the effects (a) and (b) and screening a sample having both of the effects.

In a preferred embodiment, this method further entails comparing effects (a) and (b) obtained for the candidate compound to those obtained for (Z)-N-[2-methoxy-5-[2-(3,4,5-trimethoxyphenyl)vinyl]phenyl]-L-serinamide hydrochloride; and identifying candidate compounds in which at least one of effects (a) and (b) are equal to or greater than those obtained with (Z)-N-[2-methoxy-5-[2-(3,4,5-trimethoxyphenyl)vinyl]phenyl]-L-serinamide hydrochloride.

The invention corresponds to a method of specifically selecting or screening a substance having the effects (a) and (b) in the administration to a tumor-bearing living body. Accordingly, regarding this invention, all the descriptions on the invention of the invention drug(s) can be referred to.

Especially, the effect of contracting a blood vessel preferably arises due to contracting a blood vessel to decrease or stop the tumor blood flow, and the effect of hemolyzing red blood cells is preferably an effect of hemolyzing red blood cells to clog the blood vessel of the tumor tissue.

With respect to the tumor-bearing experimental animals, rodents such as rats are preferably used because of easy procurement, keeping and handling or the like, and the administration is preferably intravenous administration.

The dose of the samples in case of rodents such as rats is preferably from approximately 0.01 to 1,000 mg/kg, more preferably from approximately 0.05 to 500 mg/kg, further preferably from 0.1 to 100 mg/kg.

A substance or a composition that is obtained or can be obtained by such a screening method is also included in the invention. However, preferably, a substance and a composition known as an anti-tumor agent have to be excluded (i.e., MTPVS).

In the other embodiment, the invention resides in a method of treating or improving a tumor, particularly a method of necrotizing the tumor, by administering a substance having the effects (a) and (b) to a tumor-bearing living body to animals, especially humans (patients), requiring treatment of the tumor.

As stated above, the effect of contracting a blood vessel is preferably an effect of contracting a blood vessel to decrease or stop the tumor blood flow, and the effect of hemolyzing red blood cells is preferably an effect of hemolyzing red blood cells to clog the blood vessel of the tumor tissue.

With respect to the type of the administration (the type for administering the substance(s)), any of the types of the tumor necrosis agents (invention drug(s)) described above can be used.

The mode for carrying out the invention is described in more detail below.

The invention includes several embodiments, namely, the tumor necrosis agents (drugs), methods of screening substances with tumor necrosis effect (function) which can be used as the active ingredients thereof, uses of the substances with the tumor necrosis effect for the production of the tumor necrosis agents, methods of treating or improving tumors and the like.

Specifically, the invention resides in use of the substances in which the following effects (effects a. and b.) are expressed in vivo in animals as the active (effective) ingredients of the drugs or the drugs using the active ingredients in this manner, uses of the drugs in the treatment or the like, methods of screening the substances (the foregoing active ingredients) having such effects and the like, for which the contents of the following effects and the confirmation (evaluation) methods thereof are important:

a. an effect of contracting blood vessels that feed nutrients to tumor tissues, and

b. an effect of hemolyzing red blood cells (erythrocytes) in blood vessels on a peripheral portion of the tumor tissue.

The blood vessels that feed nutrients to tumor tissues are not blood vessels contained in tumor tissues but blood vessels that feed blood to tumor tissues, for example, arterioles. Blood vessels just before passing blood through tumor tissues are preferable. The effect of contracting the blood vessels is, for example, an effect of reducing an inner diameter of the blood vessel, and it can easily be confirmed and evaluated by biomicroscopy.

The contraction of the blood vessels can decrease or stop the tumor blood flow resulting in limited or blocked feeding of nutrients to tumor tissues.

The blood vessels on the peripheral portion of the tumor tissues refer to blood vessels on a portion included in the tumor tissues and the blood vessels are present on an outer peripheral portion of the tumor tissues.

Hemolysis of the red blood cells in the blood vessels on the peripheral portion of the tumor tissues can easily be evaluated by biomicroscopy and comparative observation of histopathological sections of tumor blood vessels of rodents such as rats. The tumor blood vessels can be clogged by hemolysis of the red blood cells, and this effect is irreversible.

Such effects can preferably block the blood flow of tumor tissues irreversibly to necrotize the tumor tissues. Accordingly, the tumor tissues can selectively be destroyed or damaged.

However, such effects do not occur in normal tissues. The effects are, even though partially occurring, temporal (reversible), and recover easily. As a result, the substances having such effects cause little or no side effects, and they can destroy or damage the targeted tumor tissues effectively and selectively. Thus, they can fully be expected as drugs in the field of cancers.

It is not difficult to obtain the substances having such effects. For example, the substances having the effects of the present invention can be readily obtained by administering samples to experimental animals such as rats and comparing them with the control group (screening method of the invention). For example, it is possible to obtain substances that exhibit the foregoing effects with a significant difference in comparison with the control group. Preferably, substances having at least one of effects (a) and (b) that are equal to or higher than those of MTPVS can also be obtained.

The invention drugs comprising the substances having the foregoing effects as active (effective) ingredients are described.

The invention relates to, as stated above, specific drugs, tumor necrosis agents (invention drugs), methods of treatment or improvement with these drugs, uses of the substances (active ingredients) having the foregoing effects for production of these drugs, methods of screening the substances having the foregoing effects and the like. Since the drugs can include the common important contents in the invention, the invention is described in detail below upon focusing on the drugs.

The invention drugs may be desired drugs per se or pharmaceutical composition containing the same, and the object to which these are administered is not particularly limited. Animals (including humans) requiring the treatment or improvement of tumor can be mentioned as a typical example. These drugs are applied to living bodies requiring the drugs (i.e., in need thereof), especially mammals, usually humans (patients) at effective doses.

In the present invention, the methods of using the drugs and pharmaceutical compositions correspond to methods of treating or improving tumor management in a patient/subject in need thereof.

With respect to the essential active ingredients used in the invention drugs, the substances having the foregoing effects (a) and (b) are administered to experimental animals for confirmation, or such substances are selected (screening methods of the invention), and the substances confirmed thereby can be used as active ingredients. At this time, even though some of known anti-tumor agents have such effects, the desired effects are not satisfactory or are too low. It is thus preferable to use substances having the effects that are equal to or higher than those of MTPVS as will be shown in the following Examples.

The methods of administering the invention drugs and pharmaceutical compositions containing the same to patients (e.g., humans) are not particularly limited. Accordingly, various administration methods such as oral administration and parenteral administration (intravenous administration or the like) intraperitoneal administration, percutaneous administration and inhalation administration can be employed, and intravenous administration is preferable in view of efficacy.

The invention drugs have tumor necrosis effect and, as such, the present invention is not limited by the type of tumor, the degree of progression of disease, the sites where tumors develop and the like. The drugs can widely be applied to the object requiring the anti-tumor effect.

The active ingredients can solely be used as a drug. Further, they can contain various pharmacologically acceptable substances (as auxiliaries) for preparations (hereinafter also referred to as “pharmaceutically acceptable carriers”). The substances for preparations can properly be selected depending on the dosage form of the preparations. Examples thereof can include excipients, diluting agents, additives, disintegrants, binders, coating agents, lubricants, a flavor enhancer or flavor substance, sweeteners, solubilizers and the like. Specific examples of the substances for preparations can include magnesium carbonate, titanium dioxide, lactose, mannitol, other saccharides, talc, milk protein, gelatin, starch, cellulose, derivatives thereof, animal and vegetable oils, polyethylene glycol, and solvents, for example, sterile water and monohydric or polyhydric alcohols such as glycerol.

The invention drugs can be formulated into various pharmaceutical preparations including preparation forms that will be developed in future. Examples of suitable pharmaceutical preparations formulations include preparations of various administrations such as oral administration, parenteral administration, intraperitoneal administration, percutaneous administration and inhalation administration. In order to formulate drugs of the invention into such various pharmaceutical preparations, methods that have been known or will be developed in future can properly be employed.

Examples of pharmaceutical preparations include appropriate solid or liquid preparations such as granules, powders, coated tablets, tablets, (micro)capsules, suppositories, syrups, juices, suspensions, emulsions, drops, injection solutions and preparations for prolonging release of active substances (i.e., a slow-release form).

It is a matter of course that the invention drugs in the form of the preparations listed above have to contain the active ingredients in an amount effective to bring forth pharmaceutical effect.

The dose of the tumor necrosis agent used in the drug of the invention (invention drug) is appropriately selected depending on the degree of the effects of the active ingredients, the degree of progression of diseases, the form of the preparations and the like. For example, the preparation using the active ingredient that is equal in effect to MTPVS can be administered to one patient orally in a day at a dose of, preferably from approximately 1 mg to 10 g, more preferably from approximately 2 mg to 5 g, further preferably from approximately 4 mg to 4 g in terms of a net weight of the active ingredient. In case of the grave condition, the dose can further be increased. With respect to the number of administrations and the timing of the administration, it can be administered once for a few days, or several times a day. In case of parenteral administrations such as intravenous administration, the dose may be from one tenth to one twentieth (i.e., 0.05 mg to 1 g, preferably 0.1 mg to 0.5 g, more preferably 0.2 mg to 0.4 g) the dose in case of the oral administration.

The methods of using the invention drugs which is used in the methods of treating or improving tumor in the invention, especially the methods of necrotizing tumor, and so forth can readily be understood from the foregoing description, and these methods can easily be practiced.

The methods of screening the substances having the foregoing effects can easily be practiced by confirming the effects (effects (a) and (b)) using appropriate experimental animals, for example, rodents such as rats.

The effects (a) and (b) and the methods of confirming the same are as described above, and the screening methods of the invention can easily be practiced by utilizing the very description and known techniques.

Despite the fact that (Z)-N-[2-methoxy-5-[2-(3,4,5-trimethoxyphenyl)vinyl]phenyl]-L-serinamide hydrochloride (i.e., MTPVS) is excluded by proviso from the scope of identified invention drugs claimed herein, the present invention provides methods for: (a) contracting blood vessels and (b) hemolyzing red blood cells, by administering to a patient in need thereof an effective amount of MTPVS.

The above written description of the invention provides a manner and process of making and using it such that any person skilled in this art is enabled to make and use the same, this enablement being provided in particular for the subject matter of the appended claims, which make up a part of the original description.

As used above, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

Materials and Methods

Experimental Animals:

Donryu rats (Crj-Donryu; supplied by Charles River Japan, Inc.), male.

Tumor cells:

Rat lung carcinoma, Sato lung carcinoma (SLC)

Test sample:

(Z)-N-[2-methoxy-5-[2-(3,4,5-trimethoxyphenyl)vinyl]phenyl]-L-serinamide hydrochloride (MTPVS) having the following structural formula (1) was used as a test sample.

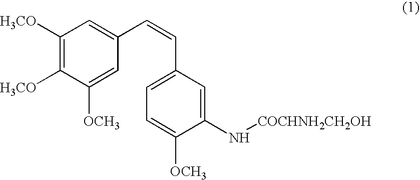

Preparation of a Test Sample:

MTPVS powder was dissolved in physiological saline to a final concentration of 10 mg/ml. The solution (10 mg/kg) was administered through the tail vein of rats. Pentobarbital salt (Nembutal; manufactured by Abbott Laboratories) and enflurane (Ethrane; manufactured by Abbott Laboratories) was used as an anesthesia. Pentobarbital was intramuscularly administered at a dose of 25 mg/kg, 10 minutes prior to the experiment, and a suitable amount thereof was additionally administered at intervals of 90 minutes to maintain anesthesia during the experiment. The concentration of enflurane was maintained at 1% (1 L/min) using an anesthesia device for small animals.

Experimental Methods:

The change of the tumor blood flow (hereinafter referred to as “TBF”) was observed through a biomicroscope. The dorsal skin of the rat was held with a transparent chamber under sterile conditions, and a small fragment of tumor was implanted thereon. After the tumor started to grow thereto, the rat was tested as described in Jpn. J. Cancer Res. 81: 279-288, 1990, which is incorporated herein by reference. The chamber comprises two titanium flames of the same shape each having a circular quartz glass window with a thickness of 300 μm. After anesthesia, the rat with the chamber was put on a stage heated at 34.5° C. in the microscope.

MTPVS used in a test group was administered through the tail vein by infusion pump. TBF and histological changes of tumor were directly observed through an optical microscope (Eclipse E800; manufactured by Nikon Corporation) with an eye lens of 10× and an object glass of 2× to 20×. The blood vessels of tumor in the chamber were visualized with a halogen lump of 12 V and 100 W. The images of the microscope were recorded using a CCD video camera (CS-900; manufactured by Olympus Corporation), a television monitor and an S-VHS video recorder.

### Example 1

Anti-Tumor Effect Against Tumor Grown in the Transparent Chamber

The SLC tumor grown in the transparent chamber was necrotized within 25 hours after administration of 10 mg/kg of MTPVS, and this state was then maintained for 48 hours after treatment.

### Example 2

Biomicroscopic Observation of the Change of Tumor Microcirculation

In the tumor, the arteriole supplying nutrients to the tumor were markedly contracted by intravenous administration of 10 mg/kg MTPVS. With the contraction of the arteriole, the tumor blood flow was gradually decreased, and stopped almost completely 30 minutes after MTPVS administration. At this time, in the blood vessel on the peripheral portion of the tumor, the plasma concentration was decreased, and large quantities of red blood cells were retained. These red blood cells were hemolyzed within 2 to 3 hours after MTPVS administration to form clog.

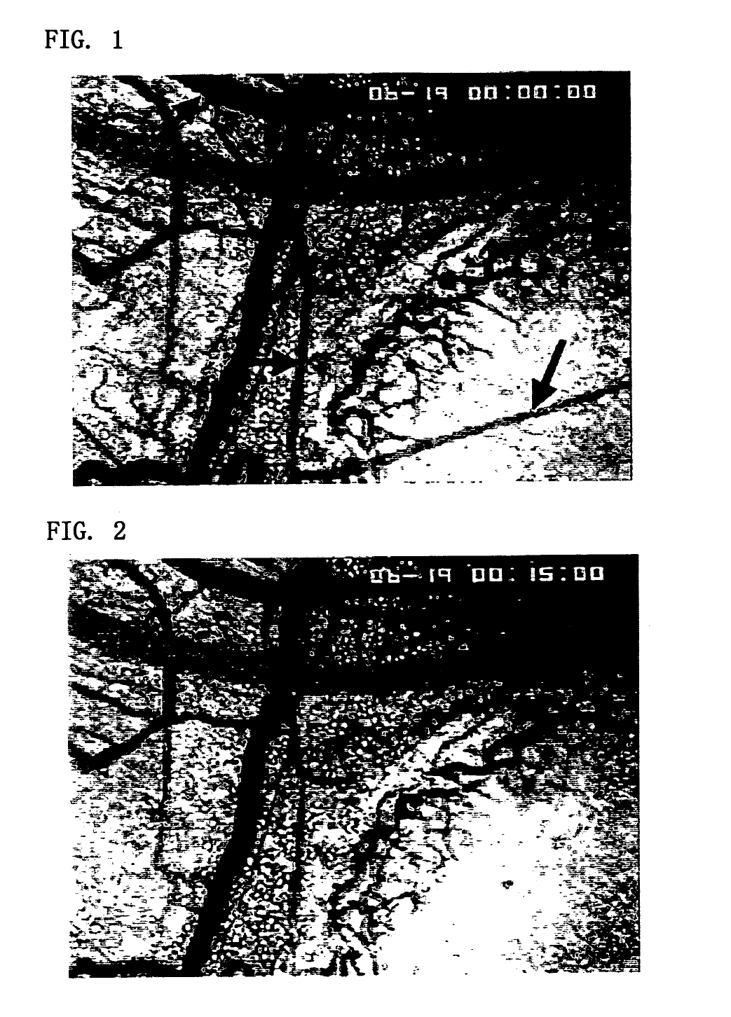

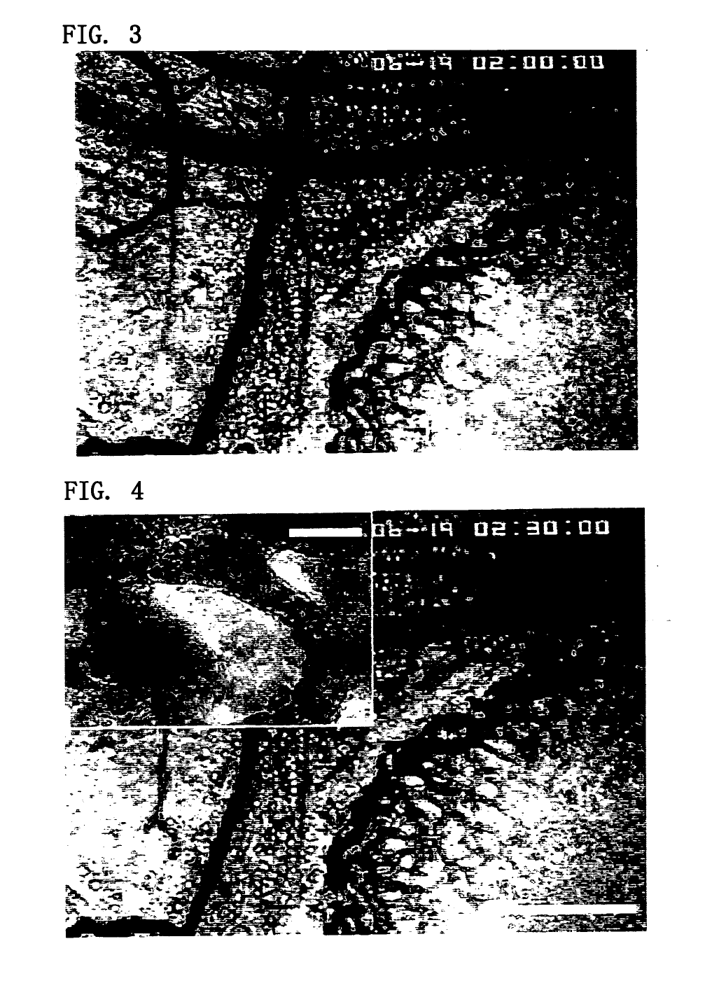

A series of these changes are shown in FIGS. 1 to 4. FIG. 1 shows observation before MTPVS administration, FIG. 2 shows observation 15 minutes after MTPVS administration, FIG. 3 shows observation 2 hours after MTPVS administration, and FIG. 4 shows observation 2.5 hours after MTPVS administration. An inserted section on the left upper portion in FIG. 4 is an enlarged view of the blood vessels on the peripheral portion of the tumor. FIGS. 1 to 4 are based on photographs taken (length of a bar: 500 μm) by using an eye lens of 10× and an object glass of 4×. The inserted section is based on a photograph taken (length of a bar: 50 μm) by using an eye lens of 10× and an object glass of 20×. It was determined that the arteriole (refer to an arrow portion in FIG. 1) that feeds nutrients to the tumor is markedly contracted by the MTPVS administration (refer to FIG. 2). Once the clog and hemolysis occurred (refer to the inserted section in FIG. 4), TBF was no longer restored.

In the normal tissues, the blood vessel structure was maintained as normal.

Based on the foregoing, it is apparent that MTPVS blocked TBF of the tumor to lead to tumor necrosis. A mechanism of blocking TBF has not been clarified. However, it has been suggested that not only the direct effect of drug active ingredient, for example, MTPVS on tumor blood vessels, but also the indirect effect via arteriole are important. Specifically, marked persistent contraction of the arteriole by administration of drug active ingredient, for example, MTPVS induced remarkable decrease of TBF at an early stage. The reason why TBF, once stopped, is no longer restored is considered to be that the red blood cells retained in the peripheral portion of the tumor are hemolyzed within 2 to 3 hours to form clogs.

The effectiveness of cancer chemotherapy based on the blocking of TBF is theoretically unrelated to the nature of cancer cells. Accordingly, the invention is expected to be useful in treatment and/or improvement of various cancers that have been so far incurable.

As an example of drugs useful for this purpose include drugs that are equal or superior to MTPVS can be mentioned.

### Advantage of the Invention

The present invention provides tumor necrosis agents that can destroy or damage tumor tissues effectively and selectively with little or no side effects. Moreover, the present invention provides methods of screening active (effective) ingredients that selectively block blood flow of tumor tissues to necrotize the tumor, uses of the active ingredients in drug(s), methods of using the drug(s), especially methods of treating or improving tumor, and the like.

Accordingly, the invention is industrially quite useful in the field of pharmaceuticals or medical care, especially cancer therapy.

Numerous modifications and variations on the present invention are possible in light of the above teachings. It is, therefore, to be understood that within the scope of the accompanying claims, the invention may be practiced otherwise than as specifically described herein.

1. A method of identifying a tumor necrosis agent having the following effects when administered to a tumor-bearing living body:
a. an effect of contracting a blood vessel that feeds a nutrient to a tumor tissue, and
b. an effect of hemolyzing red blood cells in a blood vessel on a peripheral portion of the tumor tissue,
wherein said method comprises:
administering a sample containing a candidate compound to a tumor-bearing experimental animal; and
assessing the ability of the candidate compound to provide effects (a) and (b) as compared to a control group in which the candidate compound is not administered,
with the proviso that the candidate compound is not (Z)-N-[2-methoxy-5-[2-(3,4,5-trimethoxyphenyl)vinyl]phenyl]-L-serinamide hydrochloride.

2. The method of claim 1, wherein the effect of contracting a blood vessel is an effect of contracting a blood vessel to decrease or stop tumor blood flow.

3. The method of claim 1, wherein the effect of hemolyzing red blood cells is an effect of hemolyzing red blood cells to clog the blood vessel of the tumor tissue.

4. The method of claim 1, wherein the blood flow of the tumor tissue is irreversibly blocked to necrotize the tumor.

5. The method of claim 1, wherein the living body is a mammal.

6. The method of claim 1, wherein the living body is a human.

7. The method of claim 1, wherein the experimental animal is a rat.

8. The method of claim 1, further comprising:
comparing effects (a) and (b) obtained for the candidate compound to those obtained for (Z)-N-[2-methoxy-5-[2-(3,4,5-trimethoxyphenyl)vinyl]phenyl]-L-serinamide hydrochloride; and
identifying candidate compounds in which at least one of effects (a) and (b) are equal to or greater than those obtained with (Z)-N-[2-methoxy-5-[2-(3,4,5-trimethoxyphenyl)vinyl]phenyl]-L-serinamide hydrochloride.

9. The method of claim 5, wherein effects (a) and (b) obtained with the candidate compound are equal to or greater than those obtained with (Z)-N-[2-methoxy-5-[2-(3,4,5-trimethoxyphenyl)vinyl]phenyl]-L-serinamide hydrochloride.

10. The method of claim 1, wherein said candidate compound is epinephrine.

11. A tumor necrosis agent identified by the method of claim 1.

12. The tumor necrosis agent of claim 11, wherein said agent is epinephrine.

13. A pharmaceutical composition comprising the tumor necrosis agent of claim 11 and a pharmaceutically acceptable carrier.

14. The pharmaceutical composition of claim 13, wherein the pharmaceutically acceptable carrier comprises one or more components selected from the group consisting of an excipient, a diluting agent, an additive, a disintegrant, a binder, a coating agent, a lubricant, a flavor enhancer, a flavor substance, a sweetener, and a solubilizer.

15. The pharmaceutical composition of claim 13, wherein the pharmaceutically acceptable carrier comprises one or more components selected from the group consisting of magnesium carbonate, titanium dioxide, lactose, mannitol, a saccharide, talc, milk protein, gelatin, starch, cellulose, derivatives thereof, animal oil, vegetable oil, polyethylene glycol, sterile water, a monohydric alcohol, a polyhydric alcohol, and glycerol.

16. The pharmaceutical composition of claim 13, wherein said pharmaceutical composition is in a form selected from the group consisting of a granule, a powder, a coated tablet, a tablet, a (micro)capsule, a suppository, a syrup, a juice, a suspension, an emulsion, a drop, an injection solution, and a slow-release form.

17. The pharmaceutical composition of claim 13, wherein said tumor necrosis agent is epinephrine.

18. A method of treating a tumor, comprising administering to a subject in need thereof an effective amount of the pharmaceutical composition of claim 13.

19. The method of claim 18, wherein said tumor necrosis agent is epinephrine.

20. The method of claim 18, wherein said administering is selected from the group consisting of oral administration, parenteral administration, intraperitoneal administration, percutaneous administration, and inhalation administration.

21. The method of claim 20, wherein said administering is oral and wherein said effective amount ranges from 1 mg to 10 g per day.

22. The method of claim 20, wherein said administering is parenteral and wherein said effective amount ranges from 0.05 mg to 1 g per day.

23. A method for contracting blood vessels that feed a nutrient to a tumor tissue comprising administering to a patient in need thereof an effective amount of (Z)-N-[2-methoxy-5-[2-(3,4,5-trimethoxyphenyl)vinyl]phenyl]-L-serinamide hydrochloride.

24. A method for hemolyzing red blood cells in a blood vessel of a tumor tissue comprising administering to a patient in need thereof an effective amount of (Z)-N-[2-methoxy-5-[2-(3,4,5-trimethoxyphenyl)vinyl]phenyl]-L-serinamide hydrochloride.

25. A method for clogging a blood vessel of a tumor tissue comprising administering to a patient in need thereof an effective amount of (Z)-N-[2-methoxy-5-[2-(3,4,5-trimethoxyphenyl)vinyl]phenyl]-L-serinamide hydrochloride.
